(19) United States
(12) Patent Application Publication
(10) Pub. No.: US 2024/0207006 A1
(43) Pub. Date: Jun. 27, 2024

(54) TACTILE SENSOR COVER AND PRODUCTION METHOD AND MEDICAL DEVICE

(71) Applicant: Siemens Healthcare GmbH, Erlangen (DE)

(72) Inventor: Verena SCHMIDT, Erbendorf (DE)

(73) Assignee: Siemens Healthcare GmbH, Erlangen (DE)

(21) Appl. No.: 18/519,609

(22) Filed: Nov. 27, 2023

(30) Foreign Application Priority Data
Dec. 21, 2022 (EP) 22215513.7

(51) Int. Cl. A61B 50/00; G01L 1/22

(52) U.S. Cl. CPC A61B 50/00; G01L 1/2287; A61B 2050/005

(57) ABSTRACT

One or more example embodiments of the present invention relates to a tactile sensor cover for detecting a collision with a multilayer, two-dimensional structure. The tactile sensor cover includes a reversibly deformable top layer forming an outer side of the sensor cover; a rigid base layer forming an inner side of the sensor cover; a sensor unit running between the top layer and the base layer, the sensor unit comprising two sensor layers; and a reversibly compressible spacer layer arranged between the two sensor layers, wherein at least two of the top layer, the base layer, and the two sensor layers are embodied as layers formed via an additive manufacturing technique.

### CROSS-REFERENCE TO RELATED APPLICATION(S)

The present application claims priority under 35 U.S.C. §119 to European Patent Application No. 22215513.7, filed Dec. 21, 2022, the entire contents of which are incorporated herein by reference.

Independent of the grammatical term usage, individuals with male, female or other gender identities are included within the term.

### FIELD

One or more example embodiments of the present invention relates to a tactile sensor cover for detecting a collision with a multilayer, two-dimensional structure, to a production method for the same and to a medical device comprising the tactile sensor cover.

### RELATED ART

Medical imaging systems, such as, for example an angiography device or a fluoroscopy system, but also other medical devices, are moved during the course of an examination or a treatment. The movement can comprise an adjustment of individual components of the system. For example, for capturing three-dimensional image information, a C-arm of an angiography device is swiveled over a body region of interest of the patient. In addition, the entire medical imaging system can also be moved, for example if it is a mobile device which, for an examination, firstly has to be brought into the examination surroundings. A medical imaging system can be moved in a manually, autonomously or partially autonomously controlled manner. It is an invariable requirement that neither person nor device in the immediate surroundings of the system is endangered by the movement of the imaging system. Individuals and devices in the surroundings of the system can also move. Reliable tactile sensors are therefore used nowadays for collision protection. Tactile sensors are arranged on the medical imaging system and detect contact or a collision of the system with an individual or an article in the surroundings. On the basis of the sensor signal, various safety measures can then be activated, for example a movement stop or a warning signal, for a user of the system. In further applications, tactile sensors are used to advance imaging components, for example an X-ray source, as close as possible to the patient in order to achieve improved imaging. In the future, collision monitoring or collision protection will become even more important for medical imaging systems owing to increased use of autonomous movement operation. In particular, floor- or ceiling-mounted mobile systems shall move increasingly more autonomously in the future.

Collision protection can comprise tactile sensors which detect contact, but also contactless sensors which already indicate an approach of the moving system towards an object in the surroundings. Common to contactless sensors for collision detection is that they have a dead range immediately in front of the sensor in which it is not possible to detect an approach. It is thus not possible to protect the region immediately in front of the sensor for all applications or in all situations. For example, a collision sensor system arranged on a patient couch interacting with an imaging system in the form of a magnetic resonance tomograph can be switched off following correct positioning on the gantry for image data capture. In this state, an object is positioned in the immediate vicinity in front of the collision sensor system. This object is not detected when the collision sensor system is switched on again. As a result of this limitation it is also more difficult to achieve reliable collision protection via contactless sensor systems. A further reliable tactile collision sensor is then provided in addition to the contactless sensor for this purpose. The design or market availability of standardized, reliable collision sensors is impeded accordingly.

Capacitive sensors as special embodiment variants of a contactless near-field sensor system are only suitable to a limited extent for use on mobile platforms, as are known, in particular, for mobile medical imaging systems.

Capacitive collision sensors are available in grounded and ungrounded embodiments. Grounded capacitive sensors have a detection range of up to 10 cm, but should be fixed at a defined position in the space for this, ideally should not themselves move, therefore. Ungrounded capacitive sensors, by contrast, have only a very limited detection range of approx. 1 cm to 2 cm and can therefore implement only a very limited safety zone around the medical imaging system, which is too low for many medical applications. A further challenge presented by capacitive sensors when used in a medical setting is that they have to be calibrated at the site of operation for an optimum result. Changing surroundings such as additional medical devices, infusion stands and/or different table fittings, can influence the calibration result, and this is accompanied by a high conceptual design or service input because, ultimately, the surroundings of every system is different. In case of doubt, a capacitive collision sensor system makes for all too frequent movement stops, so examination processes are disrupted and delayed.

Tactile collision sensors are known, for example, in the form of miniaturized switching strips integrated in the housing (outside: soft layer, inside: harder layer) or in the form of painted tactile housing parts in which, in the event of a collision, due to deformation of a soft cover layer forming the outer side, an electrical contact with an inner hard cover layer is closed by bridging a spacer layer located therebetween or a resistance is changed. These solutions are currently very expensive for the following reasons:

Connecting or joining hard and soft cover parts is laborious. Owing to the different hardness requirements, the two parts are manufactured separately from each other and then have to be joined. This results in undesirable variations in dimension and changes in shape, for example due to a drying, shrinking or curing process which follows manufacturing. Accordingly, the individual parts often have to be manually and laboriously post-processed. The larger these cover parts are, the more pronounced this effect is. In addition, edges and joins, which are unsightly or disadvantageous for hygiene reasons, can remain between the individual cover parts. Post-processing of the entire cover part surfaces, for example grinding or painting, can likewise be necessary in order to adhere to hygiene standards.

Tactile collision sensors made of textiles have also been used to date in the field of fluoroscopy. They are subject to severe limitations, however, in view of their three-dimensional malleability and their surface quality. Use of a textile as the sensor carrier material means that textile sensors are rarely dimensionally stable. In addition, a textile surface quality is typically structured or uneven and therefore easily soiled and difficult to clean. Compared to the tactile sensors with harder cover parts, the textile tactile sensors also have a greater structural height, and this can adversely affect the footprint of the system.

### SUMMARY

By contrast, one or more example embodiments of the present invention provide tactile sensors, which are optimized in view of their dimensional stability, the manufacturing complexity and the production costs.

A tactile sensor cover for detecting a collision as well as a production method for the same as claimed in independent claims is provided. A medical device comprising the tactile sensor cover as claimed in a further independent claim is also provided. Preferred and/or alternative, advantageous embodiment variants are the subject matter of the dependent claims.

Inventive solutions will be described below in respect of the claimed method and in respect of the claimed apparatuses. Features, advantages or alternative embodiments mentioned in this connection can likewise be transferred to the other claimed subject matters, and vice versa. In other words, concrete claims (which are directed, for example, at a method) can also be developed with features, which are described or claimed in connection with one of the apparatuses. The corresponding functional features of the method are embodied by corresponding concrete modules or units.

### BRIEF DESCRIPTION OF THE DRAWINGS

Properties, features and advantages of this invention and the manner in which they are achieved will become clearer and more comprehensible in conjunction with the following description of the exemplary embodiments, which are explained in more detail in conjunction with the drawings. This description does not limit the invention to these exemplary embodiments. Identical components are provided with identical reference characters in the different figures. As a rule, the figures are not to scale. In the drawings:

FIG. 1 shows a cross-sectional view of a tactile sensor cover in one exemplary embodiment of the invention,

FIG. 2 shows a cross-sectional view of a tactile sensor cover in another exemplary embodiment of the invention,

FIG. 3 shows a plan view of the tactile sensor cover of FIG. 1,

FIG. 4 shows a detailed view of a tactile sensor cover in another exemplary embodiment of the invention,

FIG. 5 shows a detailed view of a tactile sensor cover in a further exemplary embodiment of the invention,

FIG. 6 shows a cross-sectional view of a tactile sensor cover in a further exemplary embodiment,

FIG. 7 shows a view of a medical device according to an exemplary embodiment of the present invention,

FIG. 8 shows a schematic representation of an inventive method according to an exemplary embodiment of the present invention, and

FIG. 9 shows a schematic representation of an inventive method according to another exemplary embodiment of the present invention.

### DETAILED DESCRIPTION

One or more example embodiments of the present invention is directed in a first aspect at a tactile sensor cover for detecting a collision. The tactile sensor cover has a multilayer, two-dimensional structure. This means that the sensor cover is composed of a plurality of individual layers and, in at least one spatial direction, has a much lower, in particular flatter structure than in the two remaining spatial directions. The cover does not have to be two-dimensional in one plane, however. Instead it can assume any three-dimensional free-form. In other words, the individual layers of the sensor cover are flat in embodiments, substantially two-dimensional therefore (disregarding the structural height), or the individual layers can be curved, exhibit a three-dimensional course therefore. In this respect, each individual one of the layers of the sensor cover, in at least one spatial direction, also has a much lower, in particular flatter structure than in the two remaining spatial directions.

In embodiments, at least two layers can have the same thickness/height. In other embodiments, all layers can have the same height. In further embodiments, all layers of the sensor cover can have a mutually different thickness. Inventively, embodiments are also conceivable in which at least one layer has a variable height over its surface, one layer is not uniformly thick at each point therefore.

The sensor cover can consequently have a free-form in which all layers are uniformly thick. Alternatively, the sensor cover can have a free-form in which different layers have a different thickness. A different pressure sensitivity of the sensor cover may be achieved thereby in a spatially resolved manner. A greater external force is consequently required at some points than at other points of the sensor cover in order to trigger the sensor.

The layer-like structure of the sensor cover is formed by
a reversibly deformable top layer forming an outer side of the sensor cover,
a rigid base layer forming an inner side of the sensor cover,
a sensor unit running between top layer and base layer comprising
   two sensor layers, and
a reversibly compressible spacer layer arranged between the two sensor layers.

Top and base layers consequently form an external delimitation for the sensor cover. In embodiments, further layers can be provided. For example, a further spacer or buffer layer can be provided on the outer side of the base layer opposing the sensor layer. Additional layers can also be incorporated between said layers.

The tactile sensor cover is characterized in that at least two layers are embodied as layers formed via an additive manufacturing technique. Additive manufacturing describes and includes manufacturing methods in which three-dimensional workpieces are produced in a layer-like manner from a starting material or a material, optionally comprising different materials. Different starting materials and different methods of additive manufacturing, for example 3D printing, can be used here. Additive manufacturing offers great advantages in view of the flexibility of shaping the manufactured workpieces because additive manufacturing manages without the application of casting molds or press molds or negative molds for the workpiece. Furthermore, particularly dimensionally stable workpieces can be produced via additive manufacturing. A large number of variants have become available in the meantime in respect of the starting materials which can be used.

One advantage of the inventive sensor cover is that at least two layers of the layer structure are produced via an additive manufacturing method. Particularly advantageously, all layers are produced via an additive manufacturing method. The advantages of production thus apply to all layers of the sensor cover. In this respect, the inventive sensor cover uses the advantages of additive manufacturing technology.

The tactile sensor cover is embodied to detect contact with an object, an article or an individual in the surroundings of the sensor cover. For this purpose, the sensor cover comprises a sensor unit, which is embodied to detect contact and accompanying compression of the sensor cover and to generate a sensor signal as a function of the contact. In the present case, the sensor unit comprises two sensor layers, which are embodied to cooperate in order to generate the sensor signal.

In a preferred embodiment, the sensor layers are therefore electrically conductive and comprise a metal material. In other words, the first (adjacent to the top layer) and the second (adjacent to the base layer) sensor layer are formed from a two-dimensional metal layer, for example copper. In another embodiment, the first and the second sensor layers are formed from a carrier material, for example a plastics material, in particular a thermoplastic, in which metal particles are incorporated. When the sensor unit is activated, a voltage is applied to the sensor layers. If the top layer, and at least partially also the first sensor layer, is deformed by a collision with an object and the spacer layer is compressed in such a way that the sensor layers touch each other, a current flows between the two sensor layers. This current is representative of the collision. The sensor layers act like a switching element in this embodiment.

In other embodiments of the tactile sensor cover, the spacer layer running between the sensor layers can also be conductive, for example by adding a metal powder to its starting material. Voltage is likewise applied to the sensor layers in this embodiment. Due to the compression of the spacer layer in the event of a collision, the ohmic resistance measurably changes due to the variation in the spacing between the sensor layers. This is representative of a pressure acting on the sensor cover and can be given up as a corresponding sensor signal.

As mentioned in the introduction, the sensor layers are inventively embodied to be conductive. In this respect, connecting elements have to be provided on the sensor layers for applying a voltage or for measuring a current between the sensor layers.

In a preferred embodiments of the invention, the sensor layers comprise one integrally molded electrical connecting element respectively. The connecting element is produced directly together with the sensor layers therefore, so subsequent connection of the connecting element and the sensor layer can be omitted. For example, the connecting element can be produced together with one of the sensor layers by way of metal vapor deposition, metal sintering or another additive manufacturing technique. In particular, sensor layer and connecting element are formed from the same material in this embodiment. In embodiments of the sensor cover, a connecting element projects beyond the base area of the sensor layer, for example the connecting element extends as a narrow, flat feed line perpendicularly away from one side of the sensor layer. In embodiments, base and top layers can project beyond the base area of the sensor layer and thus form a carrier or bearing surface for the connecting element, to which the connecting element can be joined directly during manufacture. In embodiments, the connecting elements can also comprise a support layer or carrier layer to sufficiently stabilize the sensor layers, for example when the sensor layers, and therewith also the connecting elements, are formed from a thin metal layer. The connecting element can have the same structural height as the sensor layer or be flatter. In further embodiments, the connecting element can also have a thicker structural height than the sensor layer. In particular when a specific connecting interface has to be recreated, the connecting element can be higher than a sensor layer. In this way, for example, known or standardized plug connectors can be used for making contact with the sensor layer, but complex connecting geometries can also be implemented. For example, jack plugs, which are known per se, can be used for connecting the sensor layers. The connecting element can have a specific connecting or contact geometry at least at the end remote from the sensor layer, for example the connecting element can be embodied to be rather flat overall, but have a round contacting element at its end. In embodiments, sensor layer and connecting element are located solely in one shared plane. In further embodiments, the connecting element can also have a three-dimensional form. In one variant, the connecting element can be led, for example, first of all perpendicularly sideways out of the sensor layer and at a certain spacing from the sensor layer, can be led upwards or downwards, for example, at a 90° angle. The connecting element as well as the sensor layer can assume three-dimensional forms, therefore, be curved therefore. In particular, the connecting element can also be adapted in such a way to the construction of the sensor cover.

In yet different embodiments, the connecting element can be embodied as a contact hole integrally attached to a sensor layer or as an integrally attached contact wire. The contact hole is embodied as a hole or recess in the sensor layer, wherein in embodiments, it can extend perpendicularly upwards or downwards through at least one of the adjoining layers, in particular the base layer. A contact pin can then be introduced into the contact hole in order to make contact with the sensor layer. A contact wire also inventively exhibits a course upwards or downwards through further layers of the sensor cover. Contact hole and/or contact wire are advantageously surrounded by an insulating layer, which shields the connecting element from the remaining layers respectively. In this way, the structure of the sensor cover advantageously enables internal cabling in respect of the sensor cover, so the footprint of the sensor cover can be advantageously retained.

In embodiments of the sensor cover which incorporate a plurality of sensor segments, which will be described below, various constructions of the connecting element can be used in a sensor cover.

Particular advantages result if the at least two layers of the sensor cover formed via additive manufacturing are embodied as layers joined during the course of additive manufacturing. In other words, the sensor cover comprises not only a plurality of layers, which are produced individually and independently of one another via an additive manufacturing technique and are subsequently connected to one another, but at least two layers within the layer structure of the sensor cover are directly connected to one another during the course of manufacture of the two layers. Firstly, at least one subsequent joining step can be eliminated in this way. In addition, chemical joining means, for example, can be omitted. Secondly, joins and irregularities in the surface of the sensor cover can be advantageously reduced or even prevented in this way. Laborious manual post-processing steps to optimize the surface or for producing a requisite dimensional stability of the respective layer, such as grinding or milling, can be omitted. In particular, the layers connected to one another can comprise the same material, but can also be constructed from different materials. A suitable choice of material should be considered for adjacent layers joined in this way. Of course only those materials with appropriate compatibility or sufficient adhesive properties can be considered here.

Particularly preferably, all layers of the sensor cover are generated via an additive manufacturing technique and are directly connected to a different one of the layers during their production.

In embodiments of the invention, at least one layer, preferably a plurality of layers, is formed from a material comprising a plastics material. Particularly the top layer, the base layer and/or the spacer layer are embodied accordingly and are predestined therewith for manufacture via an additive technique.

Plastics materials may be divided into thermosetting plastics, thermoplastics and elastomers and are made of polymers, macromolecules therefore. The molecular structure and the macroscopic properties of plastics materials may vary within wide limits due to the production method and the admixing of additives. Since thermosetting plastics are characterized by a fixed structure and long dimensional stability, they can be used, for example, for the production of the rigid base layer, which in the sensor cover serves for the application of a counter force to the compression force introduced into the sensor cover. Inventively, the base layer is rigid, is substantially inflexible therefore, and also assumes the function of a supporting structure. In this respect, the base layer can be produced, for example, on the basis of epoxy resin.

Thermoplastics also exhibit a basically fixed structure, although this may be reversibly changed by the introduction of heat. These materials also allow reversible deformability due to particular structuring of layers of the sensor cover or a variation in the material thickness or density. Thermoplastics are thus suitable, in particular, as materials for producing base layer, spacer layer and/or top layer. Typical possible thermoplastics are, for example, polyethylene, polyamide, polyethylene phthalate, polystyrene or polyvinyl chloride.

Elastomers are elastic per se and therefore suitable as a starting material for the spacer layer or the top layer since the two layers have to be embodied to be reversibly deformed and or compressible on compression in order to support the function of the tactile sensor cover. For example, rubber can be used for producing the spacer layer and/or the top layer.

In particular, X-ray-transparent and X-ray-resistant starting materials are selected for production of the layers. This means that the layers of the sensor cover do not cause, or hardly cause, signals during X-ray imaging or that the mechanical properties of the layers are not influenced by X-ray radiation. This guarantees longevity of the sensor cover and prevents unnecessary image artifacts due to the tactile sensor cover when used in a medical device in the form of a medical X-ray imaging system or X-ray radiation therapy system, which generates medical image data via X-ray radiation or treats pathological tissue of a patient.

The advantages of additive manufacturing for producing the sensor cover become particularly clear when the layers assembled via additive manufacturing are produced from the same starting material. Even if the individual layers differ in their thickness and structure, one or more example embodiments of the present invention assumes that the sensor cover is consequently particularly dimensionally stable in the long term. In addition, a production process, as will be described in more detail below, can proceed particularly easily and quickly when fewer different resources are used. In particular, one or more example embodiments of the present invention assumes that when a plurality of sensor cover layers is produced from one and the same material, only one production machine is needed, and this signifies a streamlining and a reduction in costs for the manufacturing process.

In a preferred embodiment, in particular the top layer and the base layer are formed from the same material. Particularly preferably, as mentioned in the introduction, the mechanical stability of the two layers is determined by the density of the material. Mechanical properties such as elastic deformability or rigidity can be adjusted in these embodiments of the sensor cover solely by way of a variation in the density of the material and/or by a variation in the material thickness. In particular in embodiments with the same material and with identical material density, the top layer can have a lower structural height than the base layer in order to embody the top layer to be reversibly deformable and the base layer rigid. In other embodiments, a full material can be processed for the base layer irrespective of a suitable structural height of the two layers, while air or gas cavities or pores or the like can be provided for the top layer, which can be deformed at least within certain limits, in order to reduce the material density.

In addition to the top layer, the spacer layer also has to be reversibly deformable, in particular reversibly compressible or be compressible, to be able to reliably and repeatedly detect a collision with an object. In embodiments of the tactile sensor cover, the spacer layer is therefore embodied as a knitted fabric, mesh structure, grid structure or foam structure or has these structures. Alternatively, the spacer layer comprises a large number of support beams extending between the sensor layers.

All of said structures have a three-dimensional loose structure comprising gaps, cavities and/or holes. On the one hand these ensure that a contact between first and second sensor layer closes in the event of a compression or they bring about greater mechanical deformability of the material.

In particularly preferred embodiments, the tactile sensor cover is embodied not only to capture a collision with an object but also to detect where contact has occurred in relation to the surface of the sensor cover. In other words, in embodiments, the sensor cover is embodied to detect a collision in a spatially resolved manner. For this purpose, the two sensor layers have at least two sensor segments respectively. That is, the base area of the two sensor layers is divided or segmented. A first sensor segment respectively of the first sensor layer is arranged to be congruent with a first sensor segment of the second sensor layer. Accordingly, first and second sensor segments respectively act as independent tactile sensor units. The local resolution may be increased further by the number of sensor segments of a sensor layer. In particular, sensor covers can have larger sensor segments in certain areas and accordingly have a low local resolution there. In other areas, the sensor segments can be smaller, by contrast, and provide a correspondingly high spatial resolution. The shape of the individual sensor segments can likewise vary in a manner specific to the application.

Of course, each sensor segment has a separate connecting element, as already described above, with the connecting elements of the first, second, etc. sensor segments of the first and second sensor layers respectively being connected to one another. In particular, marginal sensor segments can have connecting elements which issue perpendicularly sideways and centrally or centrally located sensor segments can comprise contact holes extending directly out of the sensor layer, upwards or downwards through further layers of the sensor cover as connecting elements.

To protect the sensor unit against external interference, in particular moisture or liquids, in a particularly preferred embodiment, the tactile sensor cover comprises a self-contained protective capsule, which extends layer-like between top layer and first sensor layer, base layer and second sensor layer and on all sides of the sensor layers respectively as well as the spacer layer. The protective capsule therefore forms a container or a protective covering for the sensor unit and spatially separates the components of the sensor unit, in particular the sensor layers, from the remaining parts of the sensor cover. The walls of the protective capsule also have a layer-like and two-dimensional structure and follow the shaping of the remaining layers of the sensor cover. In this context, the walls of the protective capsule should likewise be understood as layers of the inventive sensor cover. They can be made of the materials described above, have to have the mechanical properties as described above and can be produced, in particular, also via an additive manufacturing technique. In particular, the walls of the protective capsule can also be joined to at least one adjacent layer of the sensor cover directly in the manufacturing process and make use of the advantages described above.

In embodiments of the sensor cover, the protective capsule has openings in the side walls and/or in its top/base walls, through which the connecting elements of the sensor layers are led outside.

In a second aspect, the present invention relates to a medical device comprising an inventive tactile sensor cover.

The medical device is characterized by use in a medical environment as well as the fact that it at least comprises components which are movable, or which device is fully movable and in this respect require collision monitoring.

A medical device can be a system for medical imaging, such as a computed tomograph, an angiography system, a conventional X-ray device, a magnetic resonance tomograph, a molecular imaging system, an ultrasound device or the like. In further embodiments, the medical device can be a therapeutic system, for example a radiotherapy system or the like. In further embodiments, the medical device can also be an assistant device, in particular a robotic assistant device. These can include, for example: assistant robots, mobile ECG devices, radiation protection apparatuses or the like.

As already mentioned in the introduction, the tactile sensor cover can, in particular owing to manufacture via an additive manufacturing technique, be brought into any three-dimensional shape. In this respect, in a preferred embodiment of the medical device, the layers of the tactile sensor cover have a three-dimensional free-form corresponding to a housing shape of the medical device. In other words, the three-dimensional shape of the sensor cover is molded to or emulates a housing shape of the medical device. The tactile sensor cover consequently replaces at least one housing part of the medical device. For example, the sensor cover replaces the housing of an X-ray tube arranged on a C-arm system or of the X-ray detector likewise arranged on the C-arm system. The C-arm itself can also be encased by an inventive sensor cover which is molded to it. If the tactile sensor cover replaces a housing part of a medical device, then its top layer forms the outer side of the housing of the medical device.

A next aspect of the present invention is directed at a method for producing a tactile sensor cover. As mentioned in the introduction, the sensor cover comprises
a reversibly deformable top layer forming an outer side of the sensor cover,
a rigid base layer forming an inner side of the sensor cover, a sensor unit running between top layer and base layer comprising
   two sensor layers, and
a reversibly compressible spacer layer arranged between the two sensor layers.

The method comprises a large number of steps. A first step is directed at the manufacture of the reversibly compressible spacer layer via an additive manufacturing technique. A second step is directed at applying the first and second sensor layers to the two opposing outer sides of the spacer layer respectively. In embodiments, application comprises both the production of the sensor layers as well as the joining of the sensor layers to the spacer layer. A further step is directed at applying top layer and base layer respectively to the outer sides of the two sensor layers via an additive manufacturing technique. Application can also comprise production/manufacture of the top layer and base layer in the third step.

In this aspect, the inventive sensor cover is constructed from the inside to the outside therefore. According to this aspect, the spacer layer can be embodied in the first step as a free-foam part, in particular, which is firstly 3D-printed from a material and subsequently expanded by heating.

In a further aspect, one or more example embodiments of the present invention relates to an alternative method for producing a sensor cover as described above. In this aspect, the sensor cover is constructed layer for layer, starting from base or top layer. The direction of construction is variable here and predefined, for example, by the chosen production method. In a first step, for example, firstly the base layer is manufactured via an additive manufacturing technique therefore. In a second step, the second sensor layer is then applied to the upper outer side of the base layer. In a third step, the reversibly compressible spacer layer is applied to the second sensor layer via an additive manufacturing technique. In a fourth step, the first sensor layer is applied to the upper outer side of the spacer layer. In a final step, the top layer is applied to the outer side of the first sensor layer via an additive manufacturing technique.

If the sensor cover comprises a protective capsule, which has already been described above, at a suitable point respectively, a production method of a sensor cover comprises a further step in which the protective capsule is produced via an additive manufacturing technique and in a preferred embodiment, is simultaneously joined to at least one adjacent layer.

Basically any additive manufacturing technique can be used in embodiments of the invention for each individual layer of the sensor cover. One step of an inventive production method consequently comprises one of the following techniques of additive manufacturing: 3D printing, laser sintering, fused deposition modeling, fused filament method or multi jet modeling.

Preferably, a plurality of, particularly preferably all, layers of the sensor cover is/are produced with or in one and the same machine without the component part having to be removed in the meantime. In this way, the sensor cover can be optimized by the production process in view of dimensional stability and precision of fit.

In particular when connecting elements are provided in the form of contact holes or contact wires, the steps can also be interlaced with one another or an intermittent order can be implemented. It can also be provided that individual layers are partially manufactured, during or between the manufacture or joining of further layers.

In a further embodiment of the inventive method for producing a tactile sensor cover, applying first and or the second sensor layer to one of the adjacent layers comprises metal sintering, vapor deposition of a metal, applying a metal layer or an additive manufacturing technique with a plastics material comprising metal particles. Ecocoating presents a further alternative for manufacturing the sensor layers. The individual techniques are known per se and will not be explained in more detail therefore.

Since the sensor layers are conductive and a metal has to be processed for their production, methods other than additive manufacturing can be used in the second or fourth steps mentioned above. In particular, vapor deposition of a thin metal layer onto an outer side of an adjacent layer presents a simple possibility for producing a sensor layer.

In further embodiments of the inventive method, individual layers can be prefabricated first of all, for example via an additive technique, and then be connected to one another. For example, inventively, the top layer and the base layer can also be manufactured individually. The same applies to the sensor layers. The sensor layers can then be placed, for example, on the top and the base layers. Top layer, sensor layer and spacer layer can then be connected to one another by manufacturing and joining the spacer layer.

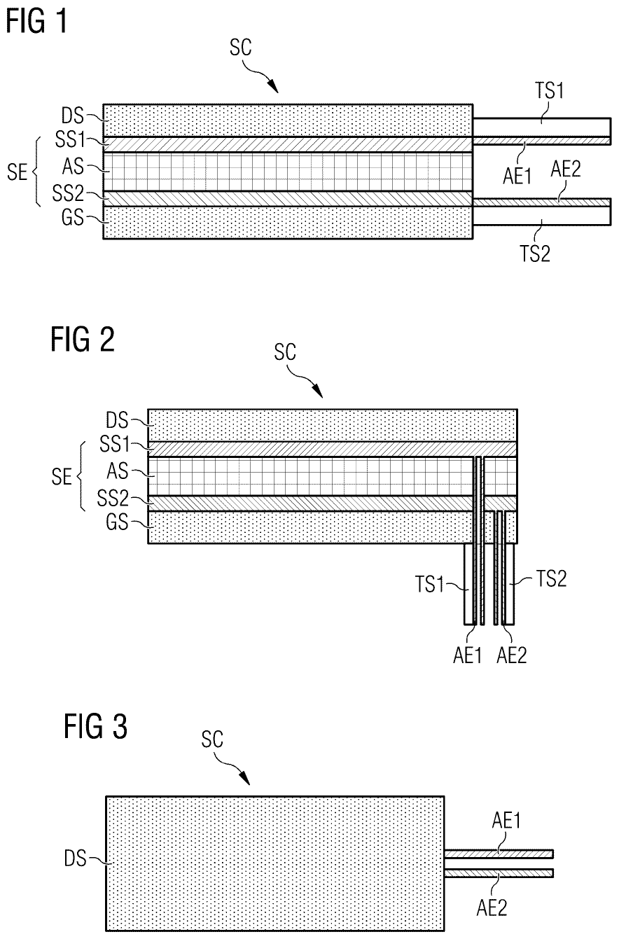

FIG. 1 shows a cross-sectional view of a tactile sensor cover SC in an exemplary embodiment of the invention.

The tactile sensor cover SC serves to detect a collision with an object in the surroundings. This can be a stationary or a mobile object, for example a person or another medical device. The sensor cover SC has a multilayer, two-dimensional structure. The sensor cover comprises a reversibly deformable top layer DS forming an outer side of the sensor cover. In the assembled state of the sensor cover Sc this is directed outwards and forms the outer side of the device on which the sensor cover SC is assembled, for example a medical device 1 as represented in FIG. 5. The top layer DS is flexible or deformable and therewith embodied to transfer a force or pressure exerted from the outside to inner layers of the sensor cover.

The sensor cover further comprises a rigid base layer GS forming an inner side of the sensor cover. This is substantially immobile and incompressible and in the event of a collision, establishes a counter force to the force exerted from the outside.

The sensor unit SE detecting a collision comprises a first and a second sensor layer SS1, SS2 running between top layer DS and base layer GS. A reversibly compressible spacer layer AS runs between the two sensor layers SS1, SS2. With deformation of the top layer DS in the event of a compression, the first sensor layer SS1, as well as the spacer layer AS, is also deformed. It is compressed.

The sensor layers SS1, SS2 are both conductive and comprise a metal material. In this embodiment, the sensor layers SS1, SS2 comprise thin, metal, conductor tracks uniformly distributed over the base area of the sensor layers SS1, SS2, which tracks are inserted in a carrier material of the sensor layers SS1, SS2. To be able to make electrical contact with the sensor layers SS1, SS2 particularly easily, the sensor layers SS1, SS2 comprise one integrally molded electrical connecting element AE1, AE2 respectively which protrude from the sensor layers respectively. The connecting elements are constructed in exactly the same way as the faces of the sensor layers and surround, for example, exactly one metal conductor track. In the present case, the connecting elements AE1, AE2 comprise a separate first and second carrier layer TS1, TS2 respectively to guarantee adequate mechanical stability of the connecting elements AE1, AE2. A voltage is applied to the sensor layers SS1, DD2 via the connecting elements AE1, AE2. The connecting elements AE1, AE2 are shown in a plan view in FIG. 3.

In the present embodiment, the spacer layer AS is formed from a porous, foam-like material. If the spacer layer AS is compressed so much that the two sensor layers SS1, SS2 touch, a current flows between the two sensor layers SS1, SS2 owing to the pores or cavities in the spacer layer. This current indicates the collision. The sensor unit SE is embodied to generate and output a corresponding sensor signal.

Inventively, at least two layers of the sensor cover SC are embodied as layers formed via an additive manufacturing technique. In particular, the top layer, base layer and spacer layer DS, GS, AS and optionally further layers, such as the protective capsule SK (cf. FIG. 6), can be considered for this because materials without specific admixtures, such as a metals, can be used for this. Materials for said layers can be chosen from the various plastics materials suitable for different applications. The embodiment of the sensor cover SC in FIG. 1 has the inventive characteristic that the at least two layers DS, GS, AS formed via additive manufacturing, for example 3D printing, are embodied as layers joined during the course of additive manufacturing. That is to say, at least one of the layers, preferably all or a plurality of layers, while they are being produced, is also immediately connected to one of its neighboring layers. The effort and costs of production are reduced in this way. In addition, owing to the use of an additive manufacturing method, the dimensional stability of the individual layers, and therewith of the entire sensor cover SC, can also be improved to meet optical and hygienic requirements.

As already mentioned, the mechanical properties of the different layers of the sensor cover SC differ. Nevertheless it is highly advantageous from a production engineering perspective if as few starting materials as possible are used for the production of a sensor cover SC. In this respect, the sensor cover SC shown in FIG. 1 has a top layer DS and a base layer GS, which are formed from the same material and in which the mechanical stability is determined or defined solely by the density of the material. In this embodiment, the two layers DS, GS are thus made, for example, from polyethylene, which is brought into the appropriate structure and shape by a suitable additive manufacturing method. Since the top layer DS has to be softer than the base layer GS or be deformable, the density of the polyethylene in the top layer DS is lower than in the base layer GS.

As mentioned in the introduction, the spacer layer AS, in particular, is also characterized in that it is elastically compressible. The compressed state can be repeatedly produced therefore. To clearly represent this mechanical property and simultaneously allow contact between first and second sensor layers SS1, SS2, the spacer layer AS is embodied as a knitted fabric. That is, the spacer layer comprises a knitted fabric, which exhibits a loose structure having air chambers. Alternatively, the spacer layer can be embodied as a component part made of plastics material and be produced via 3D printing, which emulates a knitted fabric. With an application of force, it may be compressed and subsequently returns to its original shape again. Alternatively, the spacer layer could be embodied from a mesh structure, grid structure or foam structure formed from a plastics material, all of which structures satisfy the mechanical specifications.

The sensor cover SC shown in FIG. 2 differs from the sensor cover SC shown in FIG. 1 by way of connecting elements AE1, AE2, which are embodied here in the form of downwardly led contact holes, which extend downwards in the vertical direction through the spacer layer AS, the second sensor layer SS2 and the base layer GS. Underneath the base layer the connecting elements AE1, AE2 again have carrier layers TS1, TS2 which, in this embodiment, likewise extend vertically. Inside the contact holes is a cavity, which can be used directly, for example, for making contact with the sensor layers by introducing an appropriate plug connector into the contact holes. Contact wires could be provided in an analogous manner per se instead of the contact holes. Insulating layers which are arranged sheath-like around the contact holes and separate the connecting elements AE1, AE2 from the penetrated layers of the sensor cover SC are not shown in FIG. 2.

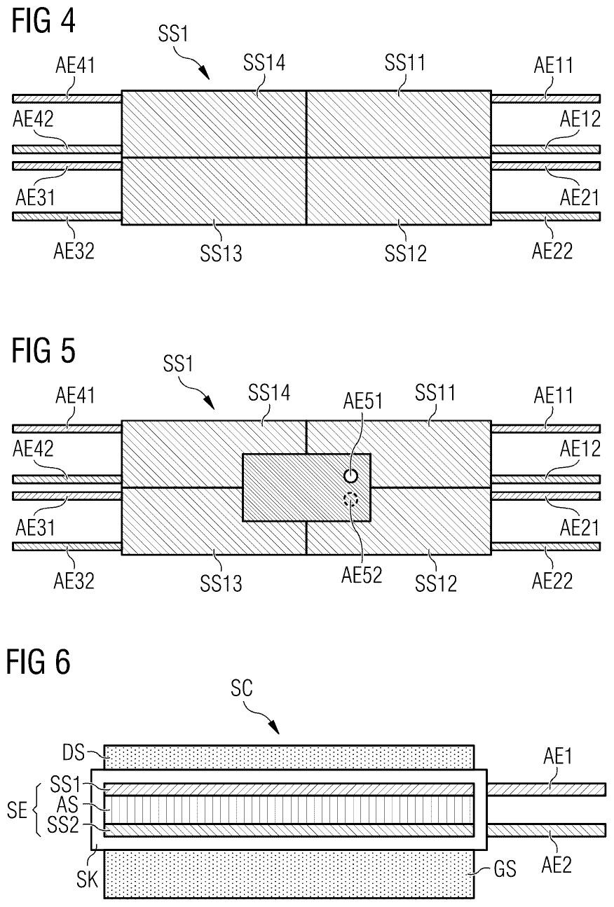

FIG. 4 shows a detailed view of a tactile sensor cover SC in another exemplary embodiment of the invention.

The sensor cover SC shown here differs from the variants shown in FIGS. 1 and 2 in that it can detect a collision with an object in the surroundings in a spatially resolved manner. The tactile sensor cover SC comprises sensor layers with sensor segments here. The sensor layer SS1, which has four equally large, rectangular sensor segments SS11, SS12, SS13, SS14 is shown. The segments of the sensor layer SS2 would exhibit an analogous shape and arrangement and are respectively concealed by the sensor layer SS1. Analogously to the segments of the second sensor layer SS2, each sensor segment SS11, SS12, SS13, SS14 has a connecting element AE11, AE21, AE31, AE41, which cooperates with the connecting elements AE12, AE22, AE32, AE42 respectively. In this way, in this embodiment, the first and the second sensor layer SS1, SS2 embody four individual collision sensors of the sensor unit SE, which can detect a collision independently of one another. Top layer DS, spacer layer AS and base layer GS can be embodied like those in FIG. 1 in this embodiment. In particular, these layers are embodied to be closed over all sensor segments.

FIG. 5 shows a detailed view of a tactile sensor cover SC in a further exemplary embodiment of the invention. Here, compared to FIG. 4, the first sensor layer SS1 comprises a further sensor segment SS15, which is arranged in the middle or centrally between the remaining four sensor segments. The remaining sensor segments SS11 to SS14 accordingly no longer have a rectangular basic shape. This shows that the sensor segments can adopt any basic shape. The second sensor layer SS2 also has a division into segments analogous to the first sensor layer SS1 here. Since the sensor segment SS15 is centrally arranged inside the first sensor layer SS1, a connecting element AE51 in the form of a contact hole is provided, which extends perpendicularly in respect of the layers. The contact hole can be embodied, for example, as described with reference to FIG. 2. A sensor segment of the second sensor layer SS2, congruent with the sensor segment SS15, accordingly has a further connecting element AE51 embodied as a contact hole.

FIG. 6 shows a cross-sectional view of a tactile sensor cover SC in a further exemplary embodiment.

The spacer layer AS of this embodiment is formed firstly from a large number of support beams extending between the sensor layers SS1, SS2. Secondly, the spacer layer AS is also conductive in this embodiment, for example by admixing metal powder, so the resistance between the sensor layers SS1, SS2 is reduced by a compression of the spacer layer AS in the event of a collision. In this way, it is possible for even instances of light contact of the sensor cover SC with sensor unit SE to be perceived and signaled. The sensor layers SS1, SS2 are embodied as planar metal layers in this embodiment, which were vapor deposited, for example, on both sides of the spacer layer AS after its production. Alternatively, the sensor layers SS1, SS2 were vapor deposited on the outer sides of the top or base layer DS, GS. Even if it is not represented in the schematic view, the connecting elements AE1, AE2 can also be vapor deposited on one of the layers respectively and be integrally connected to the sensor layers SS1, SS2.

Top and base layer DS, GS are advantageously constructed from the same material, here for example polyurethane, in this embodiment too. Schematically but not to scale, FIG. 6 illustrates that the deformability of the top layer DS was adjusted during the course of manufacturing by way of a thinner layer thickness compared to the stabile base layer.

The variant here of the tactile sensor cover SC also comprises a self-contained protective capsule SK, which extends layer-like between top layer DS and first sensor layer SS1, base layer GS and second sensor layer SS2 and on all sides of the sensor layers SS1, SS2 respectively as well as the spacer layer AS. The protective layer is preferably produced from a full material, but also via an additive manufacturing technique. Preferably, the protective capsule does not have pores, openings or the like and thus forms a protective layer for the enclosed sensor unit SE. In particular, liquids can thus be kept away from the sensor unit SE, and this has a positive effect on the life of the sensor cover SC. Depending on the choice of material for the protective capsule SK, it can also provide protection against cuts with sharp objects, for example a scalpel or the like. Of course, advantages of embodiments of the invention, which result from the application of an additive manufacturing technique, may also be transferred to the protective capsule SK. In the present case, the protective capsule SK has openings or outlets for the connecting elements AR1, AE1, or optionally more than two. Ideally, the edges of the openings do not have any gaps around the connecting elements, so penetration of a liquid is also prevented at these points.

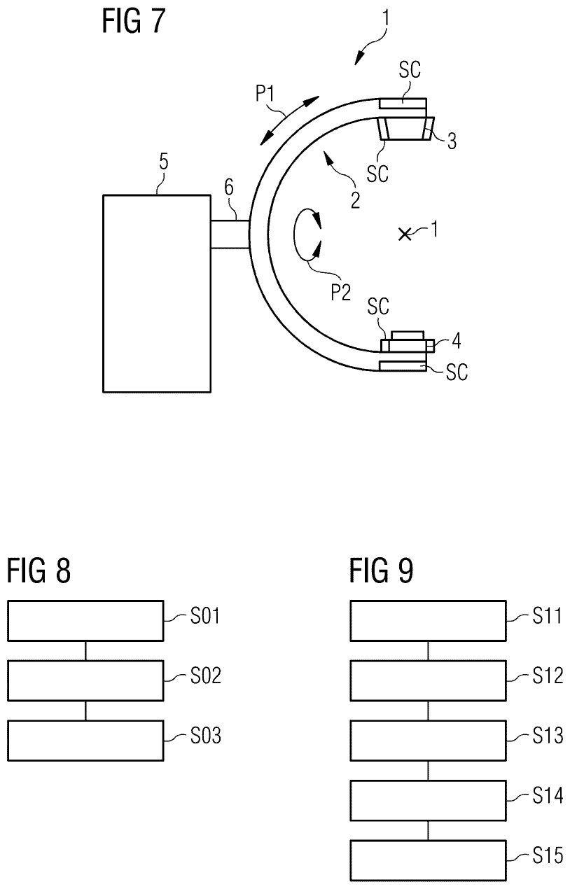

FIG. 7 shows a view of a medical device 1 according to an exemplary embodiment of the present invention.

The medical device 1 comprises a C-arm 2 on which an X-ray tube 3 and an X-ray detector 4 are arranged. The C-arm 2 is arranged on a base frame 5 to which it is coupled via a corresponding connection 6. This connection 6, which is shown here only in principle, can be, for example, a multi-articulated arm or a stand or the like. In each case, the C-arm can be moved in the space via this connection 6. Firstly, it can be moved along its arcuate path, as represented by the double arrow P1, can be moved on an orbital path therefore, so it can be rotated about the isocenter I. Furthermore, it can also be rotated about an axis, as represented by the arrow P2. In the case of an articulated arm, the C-arm 2 can also be moved around further axes, likewise in the case of a stand. FIG. 5 shows a pure schematic diagram, in order to basically represent possible movements of the C-arm. Demands on collision monitoring for the C-arm 2 result from these possible movements, in particular an orbital movement, which sometimes occurs quickly, along the arrow P1.

In this respect, on the C-arm 2, the medical device 1 comprises a plurality of inventive sensor covers SC, which can be embodied, for example, as described with reference to FIGS. 1 to 6. In particular, the X-ray tube 3 and the X-ray detector 4 and the ends of the C-arm 2 are enclosed by a sensor cover SC to protect the imaging components. Further sensor covers SC can be fitted on both the C-arm 2 and the connection 6 or the base frame 5 to achieve all-encompassing collision protection for the medical device 1. It can be inferred from FIG. 5 that the layers DS, SS1, AS, SS2, GS, etc. of the tactile sensor cover SC have a three-dimensional free-form corresponding to the housing shape of the medical device 1. Manufacture of the sensor cover SC by the application of additive manufacturing makes substantially any free-form designs of the cover conceivable. In particular, the shaping of the sensor cover can be adapted quickly and inexpensively since, for example, casting molds are no longer required for production of the layers.

As may be seen with the sensor covers SC, which are arranged at the ends of the C-arm 2, the sensor cover C is advantageously embodied as a housing part of the medical device 1 or integrated in the overall housing of the medical device 1. In this sense, the deformable top layer DS of the tactile sensor cover SC forms the outer side of the housing of the medical device 1. In this embodiment, the collision protection function can be implemented in the medical device 1 without adversely increasing the “footprint” of the device.

FIG. 8 shows a schematic representation of an inventive method according to an exemplary embodiment of the present invention for producing a tactile sensor cover SC, as described, for example, with reference to FIGS. 1 to 4.

In a step S01, firstly the reversibly compressible spacer layer AS is manufactured via an additive manufacturing technique. For example, a mesh structure of the desired size can be produced here via 3D printing. In a step S02, the sensor layers SS1, SS2 are applied to the two opposing outer sides of the spacer layer AS respectively. This can occur, in particular, by vapor deposition of a metal layer onto the two outer sides of the spacer layer. Here the inventive method takes account of the fact that a supporting layer or supporting structure, on which vapor deposition can occur over the entire surface, has to be provided on the surfaces of the spacer layer AS before vapor deposition of the metal layer in or the porous structure of the spacer layer AS. This auxiliary construction can then be, for example, washed or dissolved from the spacer layer AS in a step following manufacture. Alternatively, producing the sensor layers SS1, SS2 can also comprise metal sintering, applying a prefabricated metal layer or conductor track, or an additive manufacturing technique with a plastics material comprising metal particles. In each case, a sensor layer SS1, SS2 is directly applied to the spacer layer AS and/or attached to it. In embodiments, step S02 also comprises simultaneous vapor deposition of the connecting elements AE1, AE2. In a next step S03, the top layer DS and the base layer GS are applied to the outer sides of the two sensor layers SS1, SS2 respectively via an additive manufacturing technique. In this inventive procedure, the sensor cover SC is constructed from the inside to the outside. In one or more optional steps, a protective capsule SK can also be provided after step S02 for the sensor cover SC via an additive manufacturing technique and be joined to the sensor layers SS1, SS2 as well as to the sides of the spacer layer AS. Preferably, the steps of the production method are performed with just one additive manufacturing machine. In embodiments, some of the sensor cover can be rotated or turned in the manufacturing machine or be removed from the machine for an intermediate step and subsequently be inserted back in the manufacturing machine in order to continue manufacture according to the method. For example, after production, the spacer layer AS can be removed from the manufacturing machine for metallic vapor deposition (step S02) and be subsequently fed back to the machine for production and joining of the top and base layers DS, GS. The workpiece could be turned in the machine for joining of the top layer DS and the base layer GS. In particular, steps S01 and S03 and further optional steps, but in variants of the sensor cover SC also step S02, can comprise a 3D printing method, laser sintering, fused deposition modeling, a fused filament method or multi jet modeling respectively. The methods are known per se and will not be described in detail therefore.

In particular when the connecting elements AE1, AE2 are embodied as contact holes, all manufacturing steps S01 to S03 can comprise intermediate steps, which are directed at the production or integration of parts of the connecting elements AE1, AE2 in the respective layers. This can also comprise the manufacture of associated insulating layers and/or carrier layers TS1, TS2.

FIG. 9 shows a schematic representation of an inventive method for producing a tactile sensor cover SC according to another exemplary embodiment of the present invention. In this embodiment, the sensor cover is constructed starting from one of the sides. The structure is described starting from the base layer GS. A sequence of steps would also analogously result for a structure starting from the top layer DS.

The choice of production method is guided, for example, by the shaping of the sensor cover SC. However, it can also be determined on the basis of a specification of material in relation to one of the layers and the accompanying manufacturing technique or manufacturing machine.

In a first step S11, firstly the base layer GS is manufactured via an additive manufacturing technique therefore. In a step S12, the second sensor layer SS2 is applied to the upper outer side of the base layer GS. Subsequently in step S13, the reversibly compressible spacer layer AS is applied to the second sensor layer SS2 via an additive manufacturing technique. In a step S14, the first sensor layer SS1 is then applied to the upper outer side of the spacer layer AS. In step S15, the top layer DS is applied to the outer side of the first sensor layer SS 1 via an additive manufacturing technique.

Optional steps can also be provided in the framework of this method, in which, inserted at a suitable point, a protective capsule SK of the sensor cover SC is manufactured or joined to adjacent layers.

In addition, optional intermediate steps can be included if the connecting elements AE1, AE2 are embodied as contact holes, as described with reference to FIG. 8.

Reference is made to the statements relating to FIG. 8 with regard to the individual method steps.

Within the framework of this method it is also possible to provide that the workpiece, in particular for manufacturing the sensor layers SS1, SS2, can be removed from the manufacturing machine and be subsequently inserted again.

Although the present invention has been disclosed in the form of preferred embodiments and variations thereon, it will be understood that numerous additional modifications and variations could be made thereto without departing from the scope of the invention. For the sake of clarity, it is to be understood that the use of “a” or “an” throughout this application does not exclude a plurality, and “comprising” does not exclude other steps or elements. The expression “a number of” means “at least one”. The mention of a “unit” or a “device” does not preclude the use of more than one unit or device. The expression “a number of” has to be understood as “at least one”.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in detail by the preferred exemplary embodiment, it is not limited by this exemplary embodiment. A person skilled in the art can derive other variations herefrom without departing from the scope of the invention.

1. A tactile sensor cover for detecting a collision with a multilayer, two-dimensional structure, the tactile sensor cover comprising:
a reversibly deformable top layer forming an outer side of the sensor cover;
a rigid base layer forming an inner side of the sensor cover;
a sensor unit running between the top layer and the base layer, the sensor unit comprising two sensor layers, the two sensor layers including a first sensor layer and a second sensor layer; and
a reversibly compressible spacer layer arranged between the two sensor layers,
wherein at least two of the top layer, the base layer, and the two sensor layers are embodied as layers formed via an additive manufacturing technique.

2. The tactile sensor cover of claim 1, wherein the at least two layers formed via the additive manufacturing technique are embodied as layers joined during additive manufacturing.

3. The tactile sensor cover of claim 1, wherein the sensor layers are conductive and comprise a metal material.

4. The tactile sensor cover of claim 1, wherein the sensor layers comprise one integrally molded electrical connecting element.

5. The tactile sensor cover of claim 1, wherein the spacer layer is embodied as a knitted fabric, mesh structure, grid structure, foam structure or comprises a large number of support beams extending between the sensor layers.

6. The tactile sensor cover of claim 1, wherein the top layer and the base layer are the same material and a mechanical stability of the two layers is determined via a density of the material.

7. The tactile sensor cover of claim 1, wherein the sensor layers have at least two sensor segments respectively, wherein a first sensor segment respectively of the first sensor layer is congruently arranged with a first sensor segment of the second sensor layer.

8. The tactile sensor cover of claim 1, further comprising:
a self-contained protective capsule which extends layer-like between the top layer and a second sensor layer, the base layer and the first sensor layer and at all sides of the sensor layers and the spacer layer respectively.

9. A medical device comprising the tactile sensor cover of claim 1.

10. The medical device of claim 9, wherein the top layer, the base layer, and the two sensor layers of the tactile sensor cover have a three-dimensional free-form corresponding to a housing shape of the medical device.

11. The medical device of claim 9, wherein the top layer of the tactile sensor cover forms an outer side of a housing of the medical device.

12. A method for producing a tactile sensor cover including a reversibly deformable top layer forming an outer side of the sensor cover, a rigid base layer forming an inner side of the sensor cover, a sensor unit running between the top layer and the base layer, the sensor unit comprising two sensor layers, and a reversibly compressible spacer layer arranged between the two sensor layers, the method comprising:
manufacturing the reversibly compressible spacer layer via additive manufacturing;
applying the two sensor layers to two opposing outer sides of the spacer layer, respectively; and
applying the top layer and the base layer via additive manufacturing respectively to outer sides of the two sensor layers.

13. A method for producing a tactile sensor cover comprising a reversibly deformable top layer forming an outer side of the sensor cover, a rigid base layer forming an inner side of the sensor cover, a sensor unit running between top layer and base layer, the sensor unit comprising two sensor layers including a first sensor layer and a second sensor layer, and a reversibly compressible spacer layer arranged between the two sensor layers, the method comprising:
manufacturing the base layer via additive manufacturing;
applying the second sensor layer to the upper outer side of the base layer;
applying the reversibly compressible spacer layer to the second sensor layer via additive manufacturing;
applying the first sensor layer to an upper outer side of the spacer layer; and
applying the top layer to an outer side of the first sensor layer via additive manufacturing.

14. The method of claim 12, wherein the applying the two sensor layers comprises metal sintering, vapor deposition of a metal, applying a metal layer or an additive manufacturing technique with a plastics material comprising metal particles.

15. The method of claim 12, wherein at least the additive manufacturing of the base layer comprises 3D printing, laser sintering, fused deposition modeling, a fused filament method or multi jet modeling.
